(19) United States
(12) Patent Application Publication
(10) Pub. No.: US 2026/0147866 A1
(43) Pub. Date: May 28, 2026

(54) HEALTH MONITORING AND LOCATION TRACKING BEHAVIOR MODIFICATION SYSTEMS AND METHODS WITH DEVICE-FREE WIRELESS SENSING

(71) Applicant: Shane Joseph Crew, Pollock Pines, CA (US)

(72) Inventor: Shane Joseph Crew, Pollock Pines, CA (US)

(21) Appl. No.: 19/454,362

(22) Filed: Jan. 20, 2026

Related U.S. Application Data
(63) Continuation-in-part of application No. 18/386,201, filed on Nov. 1, 2023, now Pat. No. 12,396,659.

(51) Int. Cl. G06F 21/35; A61B 5/113; A61B 5/117; G01S 13/86

(52) U.S. Cl. CPC G06F 21/35; A61B 5/113; A61B 5/117; G01S 13/86; G01S 13/865

(57) ABSTRACT

A device-free monitoring system for a managed or secure facility uses wireless sensing nodes to transmit and receive sensing signals and to capture measurements affected by interactions with a human body. One or more processors analyze the measurements to generate outputs including one or more of presence, population count, location, physiological micro-motion metrics such as respiration and heart rate proxies, distress conditions, and identity indicators derived from device-free signatures such as gait and habitual movement. When distress, zone violations, or identity mismatches are detected, an alerting module notifies authorized staff and an access control system restricts or enables interactive interfaces such as tablets, kiosks, voice interfaces, and augmented reality or virtual reality interfaces according to facility policy. Edge processing and retention controls can minimize storage of raw measurements while preserving audit logs.

### CROSS-REFERENCE TO RELATED APPLICATIONS

This application is a continuation-in-part of U.S. patent application Ser. No. 18/386,201, filed Nov. 1, 2023, now U.S. Pat. No. 12,396,659B1, issued Aug. 26, 2025 (the ‘Parent Application’). The Parent Application is incorporated by reference in its entirety.

This application adds new subject matter directed to device-free wireless sensing for identity indicators, presence, population counting, and physiological micro-motion metrics (including respiration and heart rate proxies) in a managed or secure facility. The new subject matter is intended to operate as a standalone monitoring modality without requiring a wearable device.

A separate, co-pending U.S. patent application filed by the same applicant relates to tablet-to-band docking and charging (the “Tablet-to-Band Application”), titled “Tablet to Band Docking and Charging Method,” U.S. patent application Ser. No. 19/328,223, filed Sep. 14, 2025. The Tablet-to-Band Application is referenced for optional hybrid embodiments in which a wearable device is used in combination with the device-free wireless sensing described herein. The present application does not claim priority to the Tablet-to-Band Application, and hybrid mode is not required to practice the device-free wireless sensing inventions described and claimed herein.

### STATEMENT REGARDING FEDERALLY SPONSORED RESEARCH OR DEVELOPMENT

Not Applicable.

### FIELD OF THE INVENTION

The present disclosure relates to systems and methods for monitoring and managing dependent individuals in managed or secure environments and, more particularly, to device-free wireless sensing used to infer physiological micro-motion metrics, location, presence, population count, and identity indicators, with automated alerts and controlled access to interactive interfaces.

### BACKGROUND

Managed and secure facilities, including correctional institutions, hospitals, and long-term care facilities, commonly rely on cameras and wearable devices to support safety monitoring, accountability, and identity verification. In practice, cameras may be obstructed or raise privacy concerns, while wearables may be refused, removed, blocked, tampered with, or allowed to become uncharged.

There is a need for systems capable of detecting presence, location, distress conditions, and identity indicators without requiring an individual to wear or carry any device, while still enabling a facility to apply policy-based access control and to notify staff when intervention is warranted.

### SUMMARY

Disclosed herein are systems, methods, and computer-readable media that use device-free wireless sensing to infer, from signal variations caused by interactions with a human body, one or more operational outputs including a real-time location within a facility, a presence indication, a population count for a defined area, a physiological micro-motion metric (including respiration and heart rate proxies), a distress condition, and an identity indicator.

In some embodiments, one or more wireless sensing nodes transmit and receive radio-frequency (RF) signals (including Wi-Fi and other RF modalities) and optionally use optical/infrared and acoustic/ultrasonic sensing. Measurement features are captured and processed (optionally at an edge device) to generate inferences and confidence scores. When a distress condition, zone violation, or identity mismatch is detected, an alerting module notifies authorized staff and creates an audit log.

In some embodiments, an interface access control system dynamically enables, disables, or restricts interactive interfaces (including tablets, kiosks, environmental displays, projection interfaces, voice interfaces, and augmented reality/virtual reality interfaces) based on policy and on the device-free sensing outputs.

In optional hybrid mode, a wearable identifier or interface assignment record may be correlated with a device-free signature to improve confidence. However, the core device-free sensing functions are designed to operate without any wearable.

### BRIEF DESCRIPTION OF THE DRAWINGS

FIG. 1 is a high-level architecture diagram illustrating a managed or secure facility boundary (10), one or more wireless sensing nodes (12), one or more locator beacons (14), an optional wearable device for hybrid mode (16), a processing platform (18), an alerting module (20), a staff terminal (22), one or more interactive interfaces (24), and an interface access control system (26).

FIG. 2 is a functional block diagram of an example wireless sensing node (12) including a transmitter/receiver (34), an antenna or transducer (36), a measurement capture module (40), an edge processing module (42), and wireless sensing signals (44) transmitted and received by the node (12).

FIG. 3 is an example processing pipeline showing measurement capture (40), feature extraction (46), inference generation (48), and decision logic (50), with outputs including alerts and access-control actions.

FIG. 4 is an example facility map showing a region (52), checkpoints, authorized zones (58), prohibited zones (60), and deployment locations for sensing nodes (12) and/or beacons (14).

FIG. 5 is an example correlation diagram showing device-free signatures (132) correlated by a correlation unit (140) with optional wearable identifiers and/or interface assignment records (28), producing an identity confidence score (134).

FIG. 6 is an example alert flow including distress condition detection (74), zone violation detection (76), alert escalation (78), alert notification (80), alert acknowledgment (82), and audit logging (84).

FIG. 7 illustrates non-limiting examples of interactive interface modalities (24a-24g).

FIG. 8 illustrates a checkpoint-based identity verification scenario (e.g., medication administration) using device-free sensing (12) and an interactive interface (24), optionally supplemented by hybrid mode.

FIG. 9 illustrates a multi-modal sensing node (12) using RF modality (120), optical/infrared modality (122), and acoustic/ultrasonic modality (124), with feature extraction (46) and inference generation (48) producing multiple outputs.

FIG. 10 illustrates example device-free identity indicators including a gait signature (88), a behavior movement signature (90), an RF biometric signature (135), optional optical/audio verification (137), confidence scoring (134), and mismatch detection (146).

FIG. 11 illustrates an example access control state machine (100) including restricted state (102), special-function state (104), permitted state (106), and escalation trigger (108).

FIG. 12 illustrates privacy and edge-processing features including raw measurements (110), derived features (112), an event log (114), and a retention control module (116).

FIG. 13 illustrates additional non-limiting examples of an interactive interface (24) and/or a non-limiting interface (25), including a tablet (25a), mobile device (25b), kiosk (25c), shared terminal (25d), environmental display (25e), projection interface (25f), voice interface (25g), AR/VR interface (25h), and neural-responsive interface (25i).

FIG. 14 illustrates an example identity mismatch scenario, including an ankle band (142) and correlation outcomes (145, 146).

### DETAILED DESCRIPTION OF EMBODIMENTS

The following description is presented to enable any person skilled in the art to make and use the disclosed embodiments. Various modifications will be readily apparent, and the general principles defined herein may be applied to other embodiments without departing from the spirit and scope of the disclosure. Accordingly, the disclosure is not intended to be limited to the embodiments shown, but is to be accorded the widest scope consistent with the principles and features described.

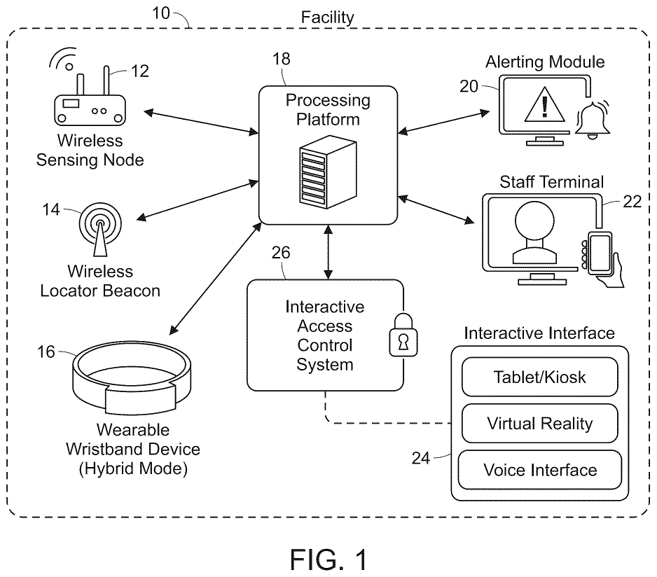

### System Overview (FIG. 1)

In an example embodiment, a managed or secure facility boundary (10) includes one or more wireless sensing nodes (12) deployed in cells, hallways, dayrooms, clinics, intake areas, sally ports, and other regions. In some embodiments, one or more locator beacons (14) are deployed for coarse location anchoring, while the wireless sensing nodes (12) provide device-free sensing for fine-grained inference.

The wireless sensing nodes (12) provide measurements to a processing platform (18) that executes feature extraction, inference generation, and policy decision logic. An alerting module (20) generates staff notifications to a staff terminal (22). An interface access control system (26) enables or restricts access to interactive interfaces (24) based on the inferred outputs and facility policy.

In optional hybrid mode, a wearable device (16), such as a wristband, ankle band, or other wearable, may provide a unique identifier or supplemental sensor data. Hybrid mode can improve confidence in identity attribution but is not required for device-free sensing.

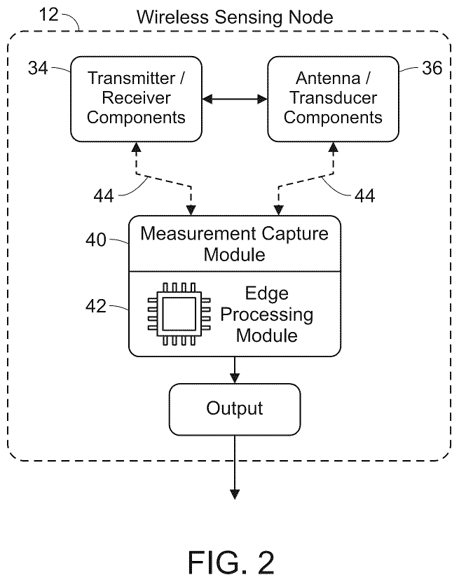

### Wireless Sensing Node Architecture (FIG. 2)

Each wireless sensing node (12) may include a transmitter/receiver (34) and an antenna or transducer (36). The transmitter/receiver (34) transmits and receives sensing signals (44) that interact with the environment and with one or more human bodies.

A measurement capture module (40) collects measurements such as channel state information (CSI), channel impulse response (CIR), received signal strength indication (RSSI), fine time measurement (FTM), round-trip time (RTT), time-of-flight (ToF), angle-of-arrival (AoA), Doppler features, multipath features, or combinations thereof, depending on the radio technology.

In some embodiments, an edge processing module (42) performs preprocessing, feature extraction, compression, anonymization, encryption, filtering, or combinations thereof before data is transmitted to the processing platform (18).

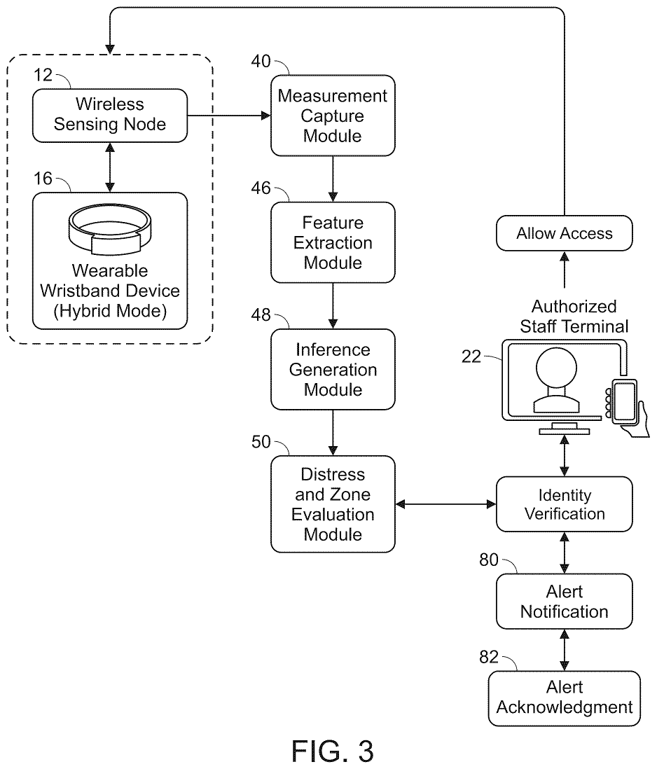

### Processing Pipeline and Outputs (FIG. 3)

As shown in FIG. 3, measurements from module (40) are processed by a feature extraction module (46) to produce derived features. An inference generation module (48) uses one or more models (statistical, machine learning, deep learning, or hybrid) to generate outputs. Decision logic (50) evaluates the outputs against facility policy to initiate alerts and access-control actions.

Non-limiting outputs include a real-time location estimate, a presence indication, a population count for a defined area, respiration rate or respiration presence metrics, heart rate proxy metrics, a distress condition, and an identity indicator.

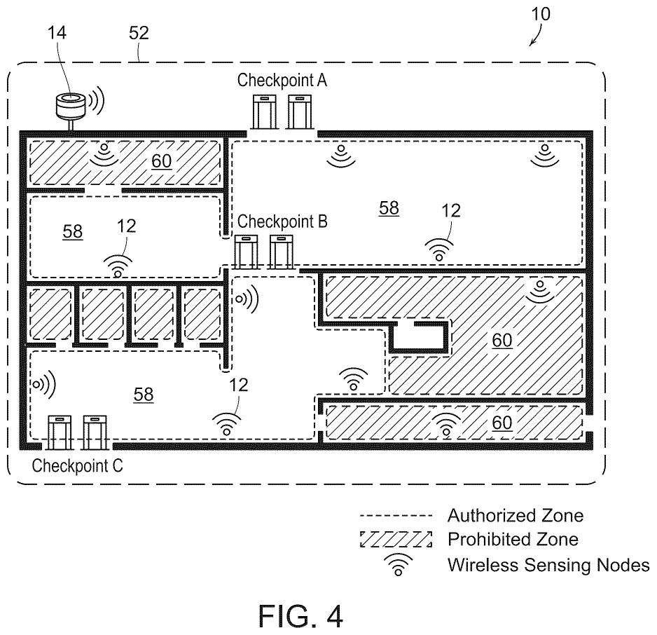

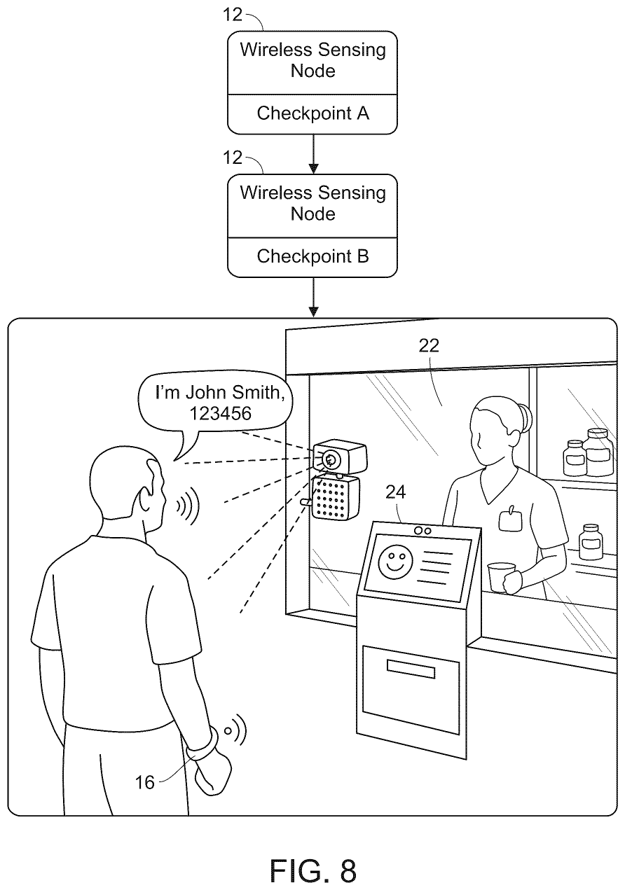

### Zone Mapping and Checkpoints (FIG. 4 and FIG. 8)

As illustrated in FIG. 4, facility regions (52) may be mapped into authorized zones (58) and prohibited zones (60). Checkpoints (e.g., A, B, C) can be defined where identity verification events occur. In one example, FIG. 8 depicts a medication administration checkpoint where an individual asserts identity via an interactive interface (24) and staff can verify the identity for safe dispensing.

Checkpoint verification can initialize, validate, or update a device-free identity profile and can be used to mitigate false attribution when multiple individuals are present in proximity.

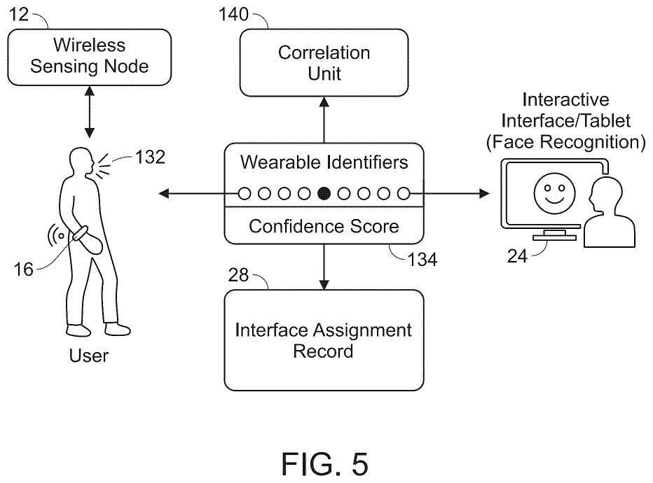

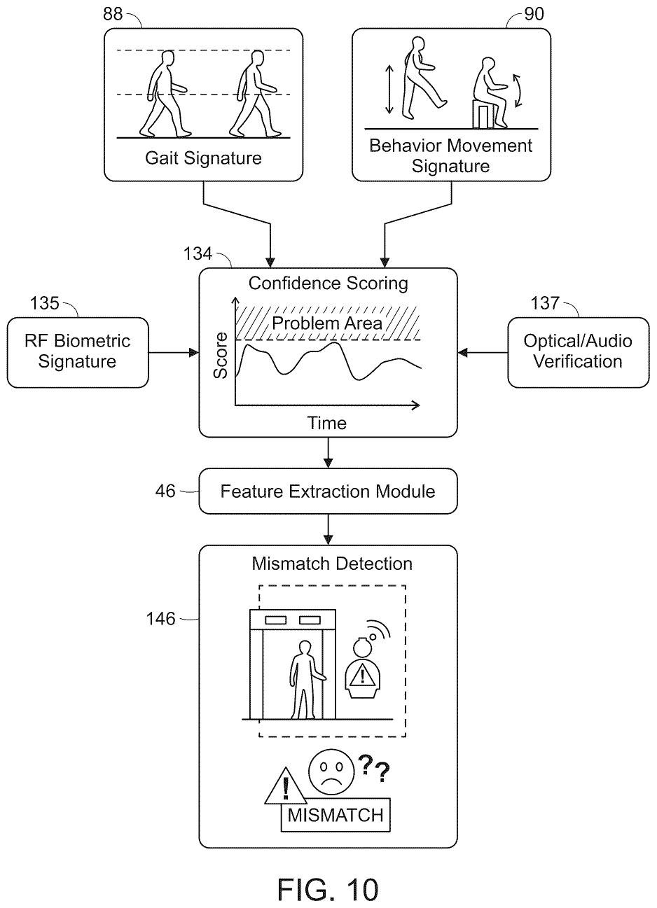

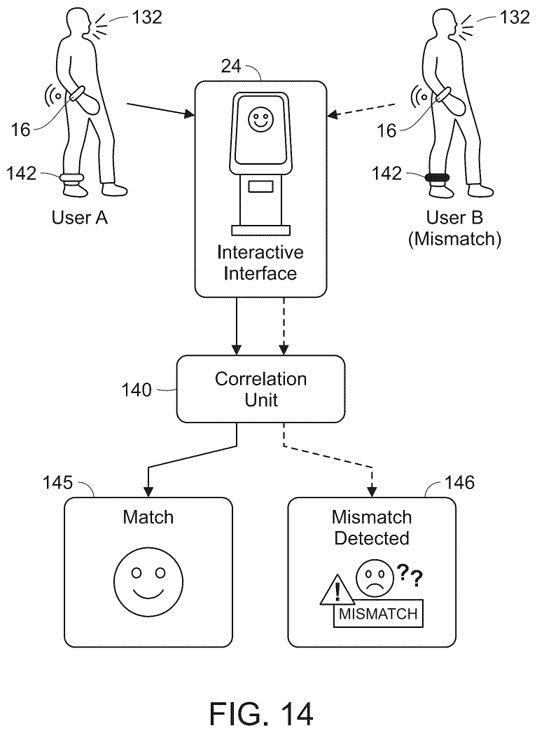

### Device-Free Identity Indicators and Correlation (FIG. 5, FIG. 10, and FIG. 14)

In some embodiments, the processing platform (18) derives device-free signatures (132) that correlate with an individual. Non-limiting examples include gait signature (88), habitual movement signatures (90), posture or micro-movement patterns, spatiotemporal behavior patterns, and RF biometric signatures (135).

As used herein, an RF biometric signature (135) may include one or more derived RF features that tend to be repeatable for a given individual over time in a given environment, such as a combination of (i) gait-related Doppler patterns, (ii) body-coupled multipath profiles, (iii) micro-motion frequency components associated with breathing, (iv) stride timing and cadence features, (v) torso and limb motion harmonics, and (vi) stable feature embeddings learned by a trained model from RF measurements.

A correlation unit (140) can correlate the device-free signature (132) with an asserted identity, with an optional wearable identifier (16), with an interface assignment record (28), or combinations thereof. The correlation unit (140) may output an identity confidence score (134) and may generate a mismatch detection event (146) when signals indicate the individual is not the asserted identity. In some embodiments, the confidence score (134) is further informed by supplemental optical/audio verification (137), such as camera-based facial verification, voiceprint verification, or other sensor-based confirmation at an interactive interface (24) or staff terminal (22).

FIG. 14 illustrates an example mismatch scenario where an assigned wearable identifier (16) and/or ankle band (142) is inconsistent with a device-free signature, causing mismatch detection (146). When consistency is detected, a match outcome (145) may be recorded.

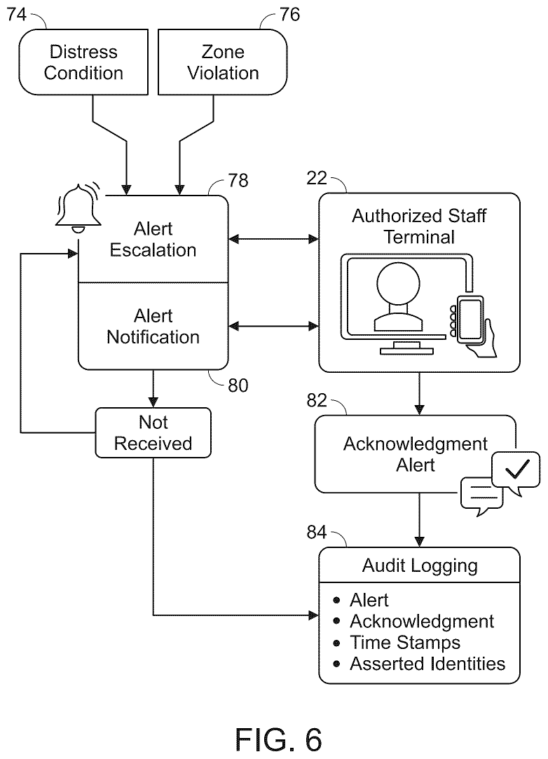

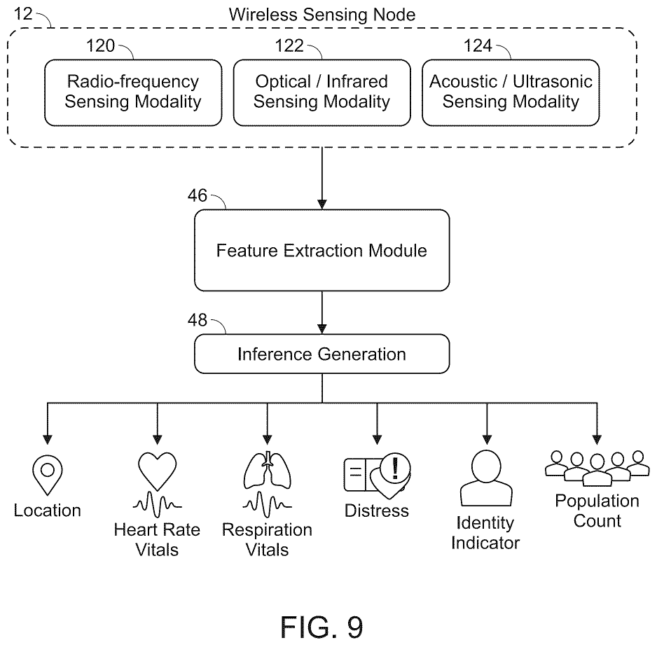

### Distress Detection and Alerting (FIG. 6 and FIG. 9)

A distress condition (74) may be inferred from absence of respiration signatures for a threshold duration, sudden falls, collapse events, prolonged inactivity, respiratory distress patterns, abnormal population count changes, or other policy-defined indicators. A zone violation (76) may be detected when an individual enters a prohibited zone (60) or exits an authorized zone (58).

The alerting module (20) can escalate alerts (78), notify staff (80), receive acknowledgment (82), and create audit logs (84). Alerts may include location indicators, confidence values, timestamp, and supporting metadata.

In some embodiments, FIG. 9 illustrates multi-modal sensing where RF modality (120) is primary, and optical/infrared (122) and acoustic/ultrasonic (124) are used to corroborate or disambiguate inferences.

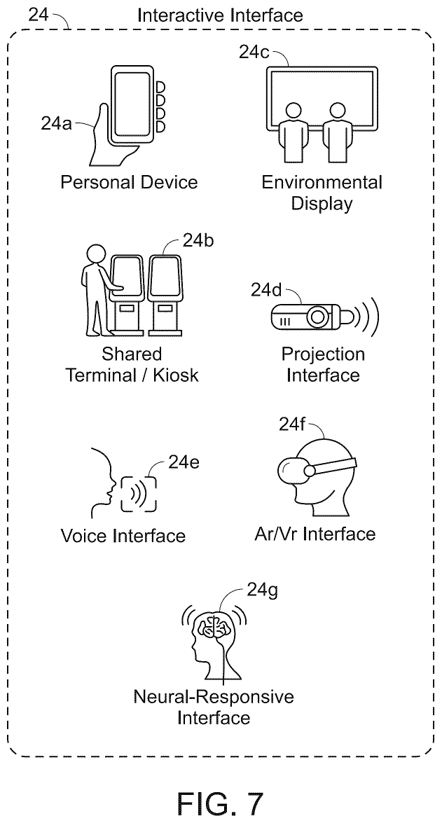

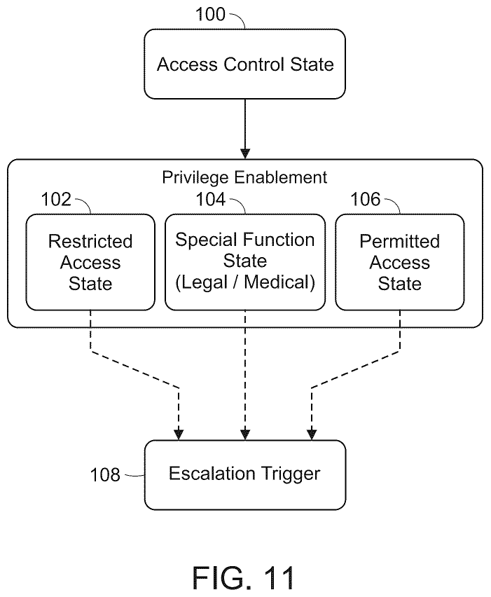

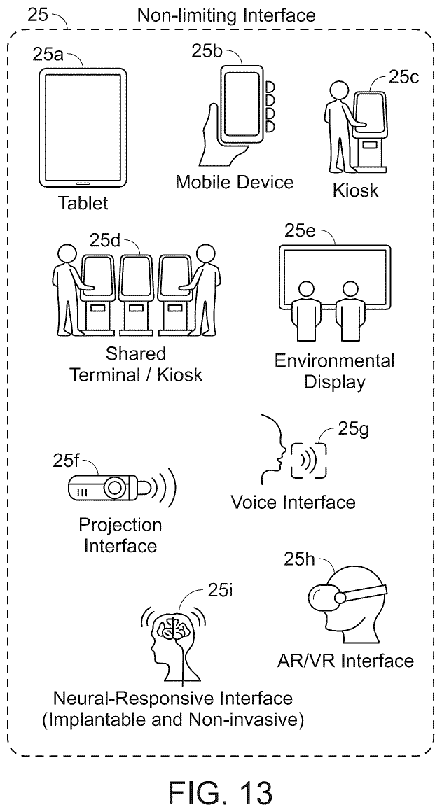

### Access Control and Interaction Modalities (FIG. 7, FIG. 11, and FIG. 13)

The interface access control system (26) can control access to interactive interfaces (24) and/or (25), including personal devices, shared kiosks, environmental displays, projection interfaces, voice interfaces, and augmented reality/virtual reality interfaces (24f).

FIG. 11 illustrates an access control state machine (100) that can apply restricted access (102), special-function access (104) (e.g., legal or medical communications), and permitted access (106). An escalation trigger (108) may be initiated when distress, zone violation, or identity mismatch occurs.

In some embodiments, access control is applied to interactions and communications rather than to a specific device. For example, the facility may enable or disable future interaction modalities (including voice and environmental interfaces) when device-free sensing indicates policy conditions are not satisfied.

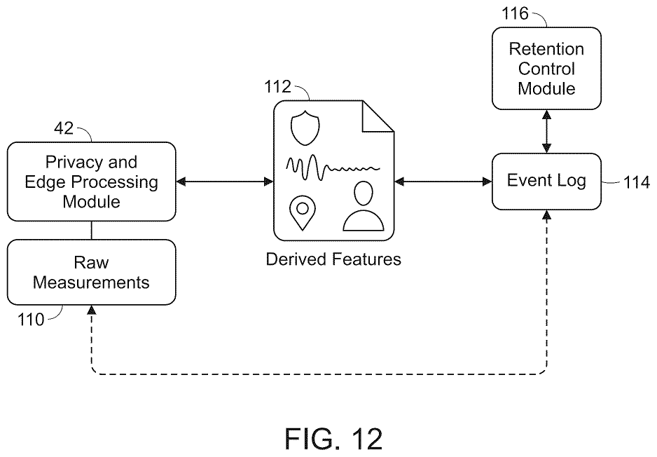

### Privacy, Data Minimization, and Retention Controls (FIG. 12)

In some embodiments, privacy and data minimization features are implemented using edge processing module (42). Raw measurements (110) may be processed into derived features (112), and only derived features and event logs (114) are retained. A retention control module (116) may enforce retention windows, access controls, encryption, and deletion policies.

In some embodiments, raw RF measurements are not stored except temporarily for calibration and are deleted after feature extraction, subject to policy and legal requirements.

### Additional Embodiments

The wireless sensing nodes (12) may be powered using Power over Ethernet and housed in tamper-resistant enclosures. The processing platform (18) may be centralized, distributed, cloud-based, on-premises, or hybrid.

The disclosure contemplates that device-free sensing and inference models may adapt over time using checkpoint validation events, supervised updates, or semi-supervised learning, while maintaining auditability and safety constraints.

1. A device-free monitoring system for a managed or secure facility, comprising: one or more wireless sensing nodes (12) deployed within a facility boundary (10), each wireless sensing node including a transmitter/receiver (34) and an antenna or transducer (36) configured to transmit and receive sensing signals; one or more processors of a processing platform (18) operatively coupled to the one or more wireless sensing nodes; an alerting module (20) configured to provide notifications to a staff terminal (22); and an interface access control system (26) configured to control access to one or more interactive interfaces (24, 25); wherein the one or more processors are configured to: capture measurements affected by interactions with at least one human body; generate, from the measurements, one or more outputs comprising at least one of (i) a presence indication, (ii) a population count for a defined area, (iii) a location estimate within the facility boundary, (iv) a physiological micro-motion metric, (v) a distress condition, or (vi) an identity indicator; and cause, based on at least one of the distress condition, a zone violation, or an identity mismatch, (a) an alert notification to the staff terminal and (b) an access-control action by the interface access control system.

2. The system of claim 1, wherein the sensing signals comprise radio-frequency signals and the measurements comprise at least one of channel state information, channel impulse response, received signal strength indication, fine time measurement, round-trip time, time-of-flight, angle-of-arrival, Doppler features, or multipath features.

3. The system of claim 1, wherein at least one wireless sensing node comprises a multi-modal node including an RF modality (120), an optical or infrared modality (122), and an acoustic or ultrasonic modality (124), and wherein at least one output is corroborated using at least two modalities.

4. The system of claim 1, wherein the physiological micro-motion metric comprises a respiration metric derived by extracting periodic components from derived features.

5. The system of claim 1, wherein the distress condition comprises at least one of suspected apnea, respiratory distress, a fall event, a collapse event, prolonged inactivity, or absence of a respiration signature above a threshold for a defined duration.

6. The system of claim 1, wherein the facility boundary is mapped into authorized zones (58) and prohibited zones (60), and wherein the zone violation comprises entry into a prohibited zone or exit from an authorized zone.

7. The system of claim 1, wherein the identity indicator comprises a device-free body-correlated signature derived from at least one of gait patterns, habitual movement patterns, posture characteristics, spatiotemporal behavior patterns, or an RF biometric signature (135).

8. The system of claim 7, further comprising a correlation unit (140) configured to maintain an identity confidence score (134) based on the identity indicator over time.

9. The system of claim 7, wherein an identity verification event is performed at a checkpoint within a facility region (52) to initialize, validate, or update an identity profile used to interpret the identity indicator.

10. The system of claim 9, wherein the checkpoint corresponds to medication administration, transport, housing movement, intake processing, or a medical procedure.

11. The system of claim 1, wherein the one or more processors are configured to detect the identity mismatch by comparing the identity indicator with an asserted identity received via an interactive interface and to generate an identity mismatch alert.

12. The system of claim 1, wherein the interface access control system controls access by at least one of enabling use, disabling use, limiting time windows, limiting features, restricting applications, restricting communications, restricting an interaction modality, or disabling a session associated with the interactive interface.

13. The system of claim 12, wherein the access-control action preserves predefined medical or legal access while restricting entertainment or recreational functions, subject to facility policy.

14. The system of claim 1, wherein at least a portion of feature extraction is performed by an edge processing module (42) to reduce bandwidth usage and to reduce retention of raw measurements (110).

15. The system of claim 14, further comprising a retention control module (116) configured to enforce retention windows for raw measurements, derived features (112), and event logs (114).

16. The system of claim 1, wherein at least one wireless sensing node is powered using Power over Ethernet and housed in a tamper-resistant enclosure.

17. The system of claim 1, wherein the interactive interfaces comprise at least one of a personal device (24a), a shared terminal or kiosk (24b), an environmental display (24c), a projection interface (24d), a voice interface (24e), an augmented reality or virtual reality interface (24f), or an interface responsive to physiological or bioelectric signals (24g).

18. The system of claim 1, wherein the access-control action is applied to interactions and communications rather than to a specific physical device, such that access control extends to future interaction modalities while device-free monitoring remains active.

19. A method of device-free monitoring within a managed or secure facility, comprising: transmitting and receiving sensing signals using one or more wireless sensing nodes; capturing measurements affected by interactions with at least one human body; processing the measurements to generate at least one of a presence indication, a population count, a location estimate, a physiological micro-motion metric, a distress condition, or an identity indicator; and responsive to at least one of the distress condition, a zone violation, or an identity mismatch, generating an alert to a staff terminal and applying an access-control action to an interactive interface.

20. The method of claim 19, further comprising performing a checkpoint identity verification event to initialize, validate, or update an identity profile used to interpret the identity indicator.

21. A non-transitory computer-readable medium storing instructions that, when executed by one or more processors, cause performance of the method of claim 19.
